(19) United States
(12) Patent Application Publication
(10) Pub. No.: US 2017/0290867 A1
(43) Pub. Date: Oct. 12, 2017

(54) EQUINE PARASITE TREATMENT METHODS AND COMPOSITIONS

(71) Applicant: SmartPak Equine LLC, Plymouth, MA (US)

(72) Inventors: Casey Fleming, Rehoboth, MA (US); Lydia Gray, Elburn, IL (US); Paal Gisholt, Duxbury, MA (US)

(21) Appl. No.: 15/482,505

(22) Filed: Apr. 7, 2017

Related U.S. Application Data
(60) Provisional application No. 62/319,718, filed on Apr. 7, 2016.

(51) Int. Cl. A61K 36/062; A23K 20/10; A23K 50/20; A61K 45/06

(52) U.S. Cl. CPC A61K 36/062; A61K 45/06; A23K 20/10; A23K 50/20

(57) ABSTRACT

New compositions and methods are provided for controlling a parasitic infection in a horse through administration of an effective amount of D. flagrans. In certain aspects, multiple, distinct strains of D. flagrans are administered. In additional aspects, the amount and treatment regimen of D. flagrans administered to a horse is based on one or more pre-identified characteristics of the horse, is combined with additional therapeutic agents for more comprehensive parasite control, or is accomplished via custom made, pre-measured daily dose packages. In another aspect, compositions and methods are provided that comprise combined use of 1) D. flagrans and 2) one or more insect growth regulators.

This Application claims the benefit of U.S. Provisional Application 62/1319,718 filed on Apr. 7, 2016. The entire contents of these applications are incorporated herein by reference in their entirety.

### FIELD

New compositions and methods are provided for controlling parasitic infection in a horse through administration of an effective amount of D. flagrans. In certain aspects, multiple, distinct strains of D. flagrans are administered. In additional aspects, the amount and treatment regimen of D. flagrans administered to a horse is based on one or more pre-identified characteristics of the horse, is combined with additional therapeutic agents for more comprehensive parasite control, or is accomplished via custom made, pre-measured daily dose packages. In another aspect, compositions and methods are provided that comprise combined use of 1) D. flagrans and 2) one or more insect growth regulators.

### BACKGROUND

Various types of parasites regularly plague horses. Intestinal parasites can pose substantial health issues in horses such as colic, weight loss and unthriftiness. Certain chemicals such as macrocyclic lactones, pyrimidines, benzimidazoles, and praziquantel have been employed as anthelmintic agents to control equine parasitic infections in horses.

These prior agents however have notable shortcomings. Parasite strains resistant to these therapeutics have evolved. Specifically, none of the three chemical classes retain full efficacy against small strongyles. There is a varying degree of resistance to each, from emerging resistance to widespread resistance. In addition, there are no new, safe, and effective anthelmintic agents likely to be approved in the near future.

It thus would be desirable to have new compositions and methods for control of equine parasitic infections.

### SUMMARY

We now provide new compositions and methods for control of parasitic infections in horses, particularly parasitic infections such as small strongyles (equine cyathostomin) and/or large strongyles infections.

In one aspect, compositions and methods of the invention comprise use and administration of effective amounts of the fungal agent Duddingtonia flagrans (D. flagrans) to a horse in need thereof.

In a preferred aspect, multiple, distinct strains of D. flagrans may be administered to a horse. Therapeutic efficacy may be enhanced by administration of multiple strains.

In a further aspect, the administered amount of D. flagrans may be pre-selected based on one or more characteristics of the horse.

In another aspect, D. flagrans may be combined with additional therapeutic agents.

In a preferred aspect, compositions and methods of the invention comprise use and administration of effective amounts of 1) the fungal agent Duddingtonia flagrans (D. fiagrans) in combination or conjunction with 2) one or more insect growth regulator agents such as cyromazine or diflubenzuron. Administration is suitably to a mammal in need thereof, such as livestock, or a horse in need thereon. In such methods, the D. flagrans and the one or more insect growth regulator agents such as cyromazine or diflubenzuron may be suitably administered substantially simultaneously of sequentially.

In the present methods, the administered D. flagrans can be administered to a horse or other subject, wherein the administered D. flagrans passes through the digestive tract of the subject and germinates or otherwise resides in the subject's manure. The D. flagrans then captures infective parasites within the subject's manure and thereby effectively controls or prophylactically treats parasite infection of the subject.

Additionally, D. flagrans may be administered in custom made, pre-measured daily dose packages. The packages may be formed from a variety of materials such as plastic or paper and include multiple sections for segregating distinct compositions being administered to a particular horse or other mammal.

Preferably, the administered amount of D. flagrans will be packaged based on one or more previously identified characteristics of a horse or other mammal. For instance, the administered amount of D. flagrans may be packaged together with additional feed and/or therapeutic agents based on one or more previously identified characteristics of the horse or other mammal.

Thus, a horse owner may utilize and administer a daily, weekly or other periodic package form of D. flagrans that has a treatment amount previously selected for the specific horse.

In particularly preferred aspects, D. flagrans may be co-administered with one or more additional therapeutic agents, for example one or more anti-parasite agents such as a macrocyclic lactone, a pyrimidine, a benzimidazole or praziquantel. The D. flagrans can be administered to the subject prior to, concurrently with, or subsequent to the administration of the one or more additional therapeutic agents.

A variety of treatment regimens may be suitably employed. For instance, D. flagrans may be administered to a horse together with food. D. flagrans may be administered to a horse under varying schedules, for example, once per day; multiple times per day; 1, 2, 3, 4 or 5 times per week; and for extended periods such as for 5, 10, 20, 30, 40, 50 consecutive weeks or more.

The invention also provides feed supplement formulations that comprise D. flagrans. The formulations may be included in a feed supplement to horses.

In a separate aspect, the invention provides a novel horse treatment composition or supplement. Certain embodiments provide that the treatment composition comprise at least two distinct strains of D. flagrans. The formulation of the invention may be in a solid or liquid form. In one embodiment, the formulation is in a pellet form.

In still another aspect, the invention provides a method of treating, preventing and/or controlling a parasite infection condition in a subject such as a horse. In certain embodiments, the method involves administering to the subject a composition or formulation described herein.

In certain embodiments, the composition or formulation of the invention is administered together with food (e.g., a feed). The administration can be an oral administration. In a further embodiment, the product is top-dressed on feed.

Other aspects of the invention include a kit which contains a D. flagrans composition or formulation described herein. The kit may come with written instructions for administration of the composition or formulation to a subject, such as, a wide variety of animals including, specifically, a horse.

Other aspects of the invention are discussed infra.

### DETAILED DESCRIPTION

In one aspect, the invention features provides novel compositions that can be used as a supplement to horses to control parasite infections.

In certain embodiments, a composition of the invention includes an effective amount of D. flagrans.

In particular aspects, a composition of the invention includes an effective amount of multiple, distinct strains of D. flagrans, for example 2, 3, 4 or more distinct strains of D. flagrans.

Exemplary suitable strains of D. flagrans include the Brazilian isolate of D. flagrans CG 768 as disclosed in A Buzatti et al., Experimental Parasitology, 159 (2015) 1-4; D. flagrans strain Troll A, CBS 101616 available from Christian Hansen AIS and as described in The EFSA Journal (2006) 334, 1-8; D. flagrans 03/99 from Argentina as described in Federica et al. Parasitol Res (2013) 112:1047-1051; and D. flagrans strain CI3 as described in Fernandez et al., Equine Veterinary Journal (1999) 31(6) 488-491.

The invention also provides a method of treating, preventing and/or controlling a parasite infection in a subject. The subject described herein is a mammal, such as a horse. Treatment, prevention and/or control methods of the invention include administering D. flagrans to a horse or other subject. The administered Dilagrans can pass through the digestive tract of the subject and reside in the subject's manure and therein capture infective parasites. This will reduce the overall parasite population within the subject's environment and thereby prophylactically treat, prevent or otherwise control parasitic infections to which the subject may be susceptible.

### Definitions

Before a further description of the present invention, and in order that the invention may be more readily understood, certain terms are first defined and collected here for convenience.

The term “administration” or “administering” includes routes of introducing a composition, formulation, or product to a subject to perform its intended function. Typically, D. flagrans will be administered orally. Other administration routes will be possible, but less typically used. In certain administration protocols, where a further treatment agent is administered together with or otherwise in conjunction with D. flagrans, the further treatment agent may be suitably administered by a variety of routes, including orally as well as an injectable (e.g. subcutaneously, intra-muscularly, intravenously) or other method. The preparations (e.g., composition, formulations, or products) are, of course, given by forms suitable for the particular administration route. For example, the preparations may be administered in tablets or capsule form, by a powder, pellets, granules or other form fed separately, or by a powder, pellets, granules or other form top dressed on food or feed.

The composition, formulations, or products can be administered alone, or in conjunction with other nutrients or therapeutic agents, or with a carrier acceptable for use in food/feed industries, or both. The composition, formulations, or products can be administered prior to, simultaneously with, or after the administration of the other nutrients or therapeutic agents.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments. “Detect” refers to identifying the presence, absence or amount of the object to be detected.

The term “effective amount” or similar term refers to the amount of an agent required to control parasite infection in an untreated subject. The effective amount of active ingredients used to practice the present invention may vary depending upon the manner of administration, the age, body weight, and general health of the subject. Such amount is referred to as an “effective” amount.

An effective amount of a component or composition delineated herein (i.e., an effective dosage) may vary widely and suitably may range from about 1×10⁴ to 1×10⁷ spores of D. flagrans per kilogram of bodyweight of the horse being treated, more typically from about 1×10⁵ to 1 or 2×10⁶ spores of D. flagrans per kilogram of bodyweight of the horse being treated.

In compositions that contain multiple, distinct strains of D. flagrans, each strain suitably will be present in an amount of at least 2, 3, 4, 5, 10, 20, 30, 40, or 50 weight percent based on total weight of D. flagrans being administered in a dose.

If desired, the dosage is administered one time per day, or multiple times per day such as twice a day. A single daily dose often will be suitable. Alternatively, the dosage is administered one time every two days, one time every three days, one time every four days, one time every five days, one time every six days, or one time per week. It will also be appreciated that the effective dosage of a composition delineated herein used for its intended purpose may increase or decrease over the course of a particular treatment.

### Compositions, Formulations and Other Products

A D. flagrans composition used herein can be in a liquid or solid form such as, a powdered, pelleted, or granulated form, or where D. flagrans spores may be coated or otherwise applied on other material such as feed.

Further, the invention provides a supplement, which includes a D. flagrans composition described herein. Such a supplement may be, for example, used as a feed supplement to a horse which comprises D. flagrans as disclosed herein. In certain embodiments, the supplement of the invention may further contain one or more additional nutrients, such as, vitamins, minerals, amino acids, proteins, herbs, fats, or combinations thereof.

The invention also provides feed supplement formulations. The formulations may be included in a feed supplement to horses.

The composition or formulation of the invention may include one or more additional biologically active and/or inactive components. Such components can be for example surfactants, disintegrants, diluents, stabilizers, preservatives, buffers, fillers, plasticizers, lubricants, excipients, and etc. Additional components that can be included in a supplement are known to those of ordinary skill in the art.

Although the present description of the invention is generally directed to use of the invention with horses, it should be understood that the invention can be used in the same manner with other animals, including pets and livestock.

### Methods

In one aspect, the invention provides a method of treating, preventing or controlling a parasitic infection in a horse. In certain embodiments, the method involves administering a composition of the invention to the horse simultaneously with or without food (e.g., a feed).

The subject is suitably a horse, but as discussed also may be other animals such as animals of livestock, including, for example, camelids (llamas and alpacas), cattle, sheep, goats, pigs, and the like, as well as pets such as dogs and cats.

The condition that can be prevented and controlled is a symptom or condition associated with a parasitic infection, including small strongyles (equine cyathostoinin) and/or large strongyles infections.

In certain embodiments, the subject could have been identified as having one or more of the diseases or disorders described herein, including a parasitic infection such as small strongyles (equine cyathostomin) and/or large strongyles infections. Identification of the diseases or disorders as described herein by a skilled veterinarian or other medical practitioner is routine in the art.

As discussed, according to another embodiment of the invention, the composition of the invention may be combined with other therapeutic agents. These therapeutic agents may be those known effective in alleviating, treating or preventing in the subject a same or different parasitic infections. It is so contemplated that such combinations may, in some cases, provide better results or synergistic effects.

It is intended that the additional therapeutic agent(s) may be administered to the subject prior to, concurrently with, or subsequent to the administration of the composition of the invention.

The therapeutic agents can be, for example, small molecule therapeutics, natural products, and/or extracts that have been known and/or used in the industry for the treatment of parasitic infections. For example, the therapeutic agent may be a macrocyclic lactone, a pyrimidine, a benzirnidazole or praziquantel. Further the therapeutic agent may be diatomaceous earth. In another aspect, the therapeutic agent could be an insect growth regulator (IGR) such as cyromazine or diflubenzuron.

Additionally, the therapeutic agent could be parasitic insects such as parasitic wasps used to control the fly population on a farm.

As discussed above, in a preferred aspect, compositions and methods of the invention comprise use and administration of effective amounts of 1) D. flagrans in combination or conjunction with 2) one or more insect growth regulator agents such as cyromazine or diflubenzuron In such methods, the D. flagrans and the one or more insect growth regulator agents such as cyromazine or diflubenzuron may be suitably administered substantial simultaneously of sequentially. In one embodiment, the D. flagrans and the one or more insect growth regulator agents such as cyromazine or diflubenzuron may be formulated together as a single or unitary dosage composition (e.g. pellets comprising each of the agents), or as separate dosage formulations that are administered at least substantially simultaneously to a subject such as a horse.

In combined but sequential administration, suitably the D. flagrans and the one or more insect growth regulator agents such as cyromazine or diflubenzuron are administered sequentially. For instance, in this embodiment, the D. flagrans is administered to a subject such as a horse first followed by administration of the one or more insect growth regulator agents such as cyromazine or diflubenzuron within a week, and more preferably within a day, including within 20, 18, 16, 12, 8, 6, 4, 2, 1, 0.5, 0.25 or 0.1 or less hours. Alternatively, one or more insect growth regulator agents such as cyromazine or diflubenzuron is administered to a subject first followed by administration of D. flagrans suitably a week, and preferably within a day, including within 20, 18, 16, 12, 8, 6, 4, 2, 1, 0.5, 0.25 or 0.1 or less

It is so contemplated that the composition of the invention and the other therapeutic agent(s) may be administered to the subject through the same route or by different routes.

In accordance with the invention, the composition can be administered to the subject once or multiple times a day. In certain embodiments, the composition is administered once, twice, three or four times a day. Single-dose daily administration often will be preferred.

### Kits and Dosages

The invention also provides a kit, which contains a composition or formulation as described above. The kit may come with written instructions for administration of the composition or formulation to a subject including, such as, a wide variety of animals. In certain aspects, the subject is an animal including a mammal (such as livestock and pets), especially, a horse.

The written instructions may also include instructions for mixing the composition to be mixed with an animal feed. Furthermore, the written instructions may generally include information for use of the composition in preventing and controlling a parasitic infection in a subject, including small strongyles (equine cyathostomin) and/or large strongyles infections.

In other embodiments, the instructions include at least one of the following: description of the one or more components of the composition; dosage schedule and administration and prevention and control of a condition to be controlled including a parasitic infection; precautions; warnings; indications; counter-indications; adverse reactions; animal pharmacology; clinical studies; and/or references. The instructions may be directly printed on a container or packaging (when present), or as a label applied to a container or other packaging, or as a separate sheet, pamphlet, card or folder supplied in or with a container or other packaging.

In particular embodiments, the kit instructions may include DFAH (Dosage Form Animal Health) product label components, such as one or more of the following: Product Facts: active ingredients Duddingtonia flagrans; inactive ingredients, as may be present in the composition; Cautions: safe use in pregnant animals or animals intended for breeding has not been proven, if animal's condition worsens or does not improve, stop product administration and consult your veterinarian; Directions for Use: give one scoop daily or other amount; Warnings: for animal use only, keep out of the reach of children and animals, in case of accidental overdose, contact a health professional immediately, this product should not be given to animals intended for human consumption.

The following non-limiting examples are illustrative of the invention.

### EXAMPLE 1

### Composition

A preferred composition contains Troll A isolate of D. flagrans spores admixed with feed supplement.

### EXAMPLE 2

### Administration

The composition of Example 1 is administered via a daily dose of the D. flagrans/feed supplement mixture daily at a dose of in an amount of 5×10⁵ spores per kg bodyweight of horse. Such daily doses continue without interruption year-round. Dosing may be curtailed during non-grazing season months if desired.

### INCORPORATION BY REFERENCE

The entire contents of all patents, published patent applications and other references cited herein are hereby expressly incorporated herein in their entireties by reference.

Those skilled in the art will recognize, or be able to ascertain using no more than routine experimentation, numerous equivalents to the specific procedures described herein. Such equivalents are considered to be within the scope of this invention and are covered by the following claims.

1. A method for treating or controlling a parasitic infection in a mammal, comprising:
administering to the mammal an effective amount of D. flagrans selected for the mammal to control a parasitic infection.

2. A method for treating or controlling a parasitic infection in a mammal comprising:
administering to the mammal an effective amount of multiple, distinct strains of D. flagrans.

3. The method of claim 1 wherein the mammal is a horse.

4. The method of claim 1 wherein the mammal is a livestock animal.

5. The method of claim 1 wherein the administered D. flagrans passes through the digestive tract of the mammal, resides in the subject's manure and therein captures infective parasites.

6. The method of claim 1 wherein the mammal has been identified as susceptible to or suffering from a small strongyles (equine cyathostomin) and/or large strongyles infection.

7. The method of claim 1 wherein the administered amount of D. flagrans has been selected based on one or more characteristics of the mammal.

8. The method of claim 1 wherein the administered D. flagrans had been packaged based on one or more previously identified characteristics of the mammal.

9. The method of claim 1 wherein the administered D. flagrans had been packaged together with additional feed and/or therapeutic agents based on one or more previously identified characteristics of the mammal.

10. The method of claim 1 wherein the one or more additional therapeutic agents are administered to the mammal in conjunction with the D. flagrans.

11. The method of claim 1 wherein the D. flagrans and/or wherein the one or more additional therapeutic agents are administered to the mammal through custom made, pre-measured daily dose packages.

12. The method of claim 9 wherein the one or more additional therapeutic agents are selected from a macrocyclic lactone, a pyrimidine, a benzimidazole, pyraziquantel, diatomaceous earth, an insect growth regulator, and/or parasite wasps.

13. The method of claim 1 wherein the D. flagrans is administered to the mammal together with food.

14. The method of claim 1 wherein the D. flagrans is administered to the mammal once per day.

15. The method of claim 1 wherein the D. flagrans is administered to the horse multiple times per day.

16. The method of claim 1 wherein the D. flagrans is administered to the horse multiple times per week for at least 30 consecutive weeks.

17. A composition for administration to a horse or other mammal comprising multiple, distinct strains of D. flagrans.

18. A packaged composition for administration to a horse or other mammal comprising an effective amount of one or more distinct strains of D. flagrans.

19. The composition of claim 17 wherein the D. flagrans is packaged in an amount based on one or more previously identified characteristics of the mammal.

20. The composition of claim 17 wherein the D. flagrans and additional feed and/or therapeutic agents are segregated within a unitary packaging.

21. A method for treating or controlling a parasitic infection in a mammal, comprising:
administering to the mammal an effective amount one or more compositions comprising: 1) D. flagrans and 2) one or more insect growth regulators.

22. The method of claim 21 wherein the one or more insect growth regulators are cyroazine and/or diflubenzuron.

23. The method of claim 21 wherein the mammal is a horse.

24. A composition for administration to a horse or other mammal comprising: 1) D. flagrans and 2) one or more insect growth regulators such as cyroazine and/or diflubenzuron.
